(19) United States
(12) Patent Application Publication
(10) Pub. No.: US 2013/0096430 A1
(43) Pub. Date: Apr. 18, 2013

(54) ULTRASONIC DIAGNOSTIC APPARATUS AND ULTRASONIC SCANNING METHOD

(71) Applicants: Hiroki YOSHIARA, Nasushiobara-shi (JP); Naohisa KAMIYAMA, Utsunomiya-shi (JP); Tetsuya YOSHIDA, Bergschenhoek (NL); Yoko OKAMURA, Irvine, CA (US)

(72) Inventors: Hiroki YOSHIARA, Nasushiobara-shi (JP); Naohisa KAMIYAMA, Utsunomiya-shi (JP); Tetsuya YOSHIDA, Bergschenhoek (NL); Yoko OKAMURA, Irvine, CA (US)

(21) Appl. No.: 13/693,536

(22) Filed: Dec. 4, 2012

Related U.S. Application Data
(63) Continuation of application No. PCT/JP2012/074256, filed on Sep. 21, 2012.

(30) Foreign Application Priority Data
Sep. 27, 2011 (JP) 2011-210995
Sep. 14, 2012 (JP) 2012-203040

(51) Int. Cl. A61B 8/08; A61B 8/14; A61B 8/00

(52) U.S. Cl. CPC A61B 8/0841; A61B 8/54; A61B 8/463; A61B 8/5207; A61B 8/5253; A61B 8/483; A61B 8/485; A61B 8/488; A61B 8/481; A61B 8/14
USPC 600/438; 600/461; 600/440; 600/453; 600/458; 600/443

(57) ABSTRACT

According to one embodiment, a detector detects a position at a leading end of a puncture needle. A determination unit determines a first scanning region in a subject and a second scanning region based on the position at the detected leading end, the second scanning region being narrower than the first scanning region. A transmission/reception controller controls a transmitter and a receiver to switches between first ultrasonic scanning for the first scanning region and second ultrasonic scanning for the second scanning region according to an instruction from an operator.

### CROSS REFERENCE TO RELATED APPLICATIONS

This application is a Continuation Application of PCT Application No. PCT/JP2012/074256, filed Sep. 21, 2012 and based upon and claiming the benefit of priority from Japanese Patent Applications No. 2011-210995, filed Sep. 27, 2011; and No. 2012-203040, filed Sep. 14, 2012, the entire contents of all of which are incorporated herein by reference.

### FIELD

Embodiments described herein relate generally to an ultrasonic diagnostic apparatus and an ultrasonic scanning method.

### BACKGROUND

An ultrasonic diagnostic apparatus can display a state of pulsation of a heart or motion of an unborn child in real time only by a simple operation to bring an ultrasonic probe into contact with a body surface, and the ultrasonic diagnostic apparatus is safe and secure. Therefore, an examination is repeatedly performed in the ultrasonic diagnostic apparatus. A system scale of the ultrasonic diagnostic apparatus is smaller than other diagnostic apparatuses, such as an X-ray diagnostic apparatus, a CT diagnostic apparatus, and an MRI diagnostic apparatus, and the ultrasonic diagnostic apparatus that can be carried in one hand has been developed. The examination can easily be performed at a bedside by the compact ultrasonic diagnostic apparatus. The ultrasonic diagnostic apparatus can also be used in an obstetric service or a home medical care, because radiation exposure is eliminated unlike the X-ray diagnostic apparatus.

Recently, an intravenous dosage type of ultrasonic contrast-enhanced medium is commercialized, and “contrast-enhanced ultrasound (CEUS)” is gradually spreading. The contrast-enhanced ultrasound is aimed at evaluation of a blood stream dynamic state such that the ultrasonic contrast-enhanced medium is injected from a vein to enhance a blood stream signal in the examinations of a heart and a liver. In many ultrasonic contrast-enhanced media, a micro bubble acts as a reflection source. For example, a second-generation ultrasonic contrast-enhanced medium called sonazoid recently put on sale in Japan is the micro bubble in which a perfluorobutane gas while a phosphorous lipid is used as a shell, and therefore a state of a back-flow of the ultrasonic contrast-enhanced medium can stably be observed by middle- and low-sound-pressure ultrasonic waves.

Applications of the ultrasonic wave are also advanced in a medical treatment. In a pathological examination of a tumor tissue, needle biopsy may be performed under an ultrasonic guide. The ultrasonic diagnostic apparatus is also used to insert a Radio Frequency Ablation (RFA) needle or to determine a treatment effect in an RFA treatment of localized tumors, such as a liver cancer. Nowadays, real-time three-dimensional scanning is also developed, and a puncture may be performed while plural sections are observed. The real-time three-dimensional scanning includes scanning in which a one-dimensional array is mechanically swung and scanning in which electronic scanning is performed using a two-dimensional array. Therefore, not only a puncture section but also a deviation of the needle in a depth direction can simultaneously be observed.

It is necessary to check a three-dimensional treatment effect and tissue information around a needle tip after the puncture. However, the three-dimensional scan is inferior to the two-dimensional scan in spatial resolution and time resolution. A scanning method for analyzing detailed information on a neighborhood of the needle is not established yet.

It is an object to provide an ultrasonic diagnostic apparatus and an ultrasonic scanning method, for being able to improve accuracy of the ultrasonography in which the needle inserted in the subject is used.

### BRIEF DESCRIPTION OF THE DRAWINGS

FIG. 1 is a view illustrating a configuration of an ultrasonic diagnostic apparatus according to an embodiment.

FIG. 2 is a view illustrating a typical flow of ultrasonography performed under control of a system controller in FIG. 1.

FIG. 3 is a view illustrating an example of a local scanning region including a center of a current position at a leading end of a puncture needle 100 in FIG. 1.

FIG. 4 is a view illustrating an example of the local scanning region including a center of a predicted reachable position at the leading end of the puncture needle 100 in FIG. 1.

FIG. 5 is a view illustrating an example of a second ultrasonic scanning performed by a transmission/reception controller in FIG. 1.

FIG. 6 is a view illustrating another example of the second ultrasonic scanning performed by the transmission/reception controller in FIG. 1.

FIG. 7 is a view illustrating another example of the second ultrasonic scanning performed by the transmission/reception controller in FIG. 1.

FIG. 8 is a view schematically illustrating switching between first ultrasonic scanning and the second ultrasonic scanning, which is performed by the transmission/reception controller in FIG. 1.

FIG. 9 is a view illustrating a display example of an image in ultrasonography by a display unit in FIG. 1.

FIG. 10 is a view illustrating an operation example by the transmission/reception controller in FIG. 1 when the second ultrasonic scanning is in an SWE mode.

FIG. 11 is another view schematically illustrating the switching between the first ultrasonic scanning and the second ultrasonic scanning, which is performed by the transmission/reception controller in FIG. 1.

FIG. 12 is a view illustrating local scanning region determining processing performed by a transmission/reception controller according to a first modification.

FIG. 13 is a view illustrating local scanning region determining processing performed by a transmission/reception controller according to a second modification.

### DETAILED DESCRIPTION

In general, according to one embodiment, an ultrasonic diagnostic apparatus includes a transducer, a transmitter, a determination unit, a determination unit, a transmission/reception controller. The transducer generates an ultrasonic wave and converts the ultrasonic wave from a subject into an echo signal. The transmitter supplies a driving signal to the transducer. The receiver performs signal processing to the echo signal from the transducer. The detector detects a position at a leading end of a puncture needle. The determination unit determines a first scanning region in the subject and a second scanning region based on the position at the detected leading end, the second scanning region being narrower than the first scanning region. The transmission/reception controller controls the transmitter and the receiver to switches between first ultrasonic scanning for the first scanning region and second ultrasonic scanning for the second scanning region according to an instruction from an operator.

Hereinafter, an ultrasonic diagnostic apparatus and an ultrasonic scanning method according to an embodiment will be described with reference to the drawings.

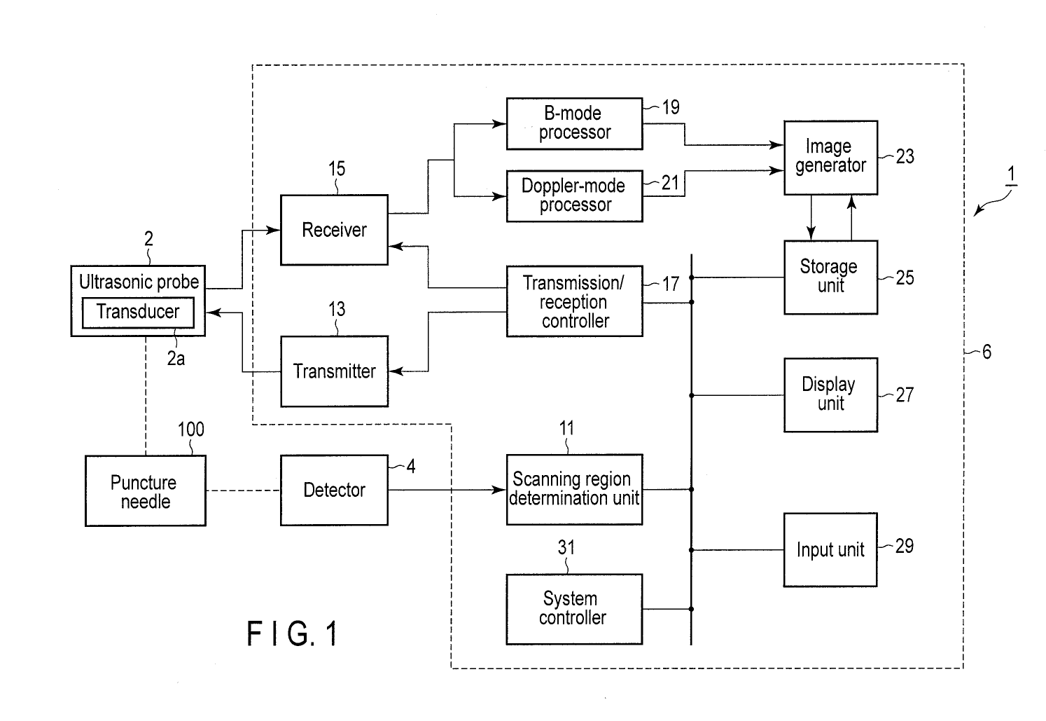

FIG. 1 illustrates a configuration of an ultrasonic diagnostic apparatus 1 according to the embodiment. As illustrated in FIG. 1, the ultrasonic diagnostic apparatus 1 includes an ultrasonic probe 2, a detector 4, and an apparatus body 6. The apparatus body 6 includes a scanning region determination unit 11, a transmitter 13, a receiver 15, a transmission/reception controller 17, a B-mode processor 19, a Doppler-mode processor 21, an image generator 23, a storage unit 25, a display unit 27, an input unit 29, and a system controller 31.

The transmitter 13, the receiver 15, and the like, which are incorporated in the apparatus body 6, may be constructed by hardware, such as an integrated circuit, and constructed by a software program that is modularized in a software manner. A function of each component will be described below.

The ultrasonic probe 2 includes plural transducers 2a that are two-dimensionally arrayed. In response to a driving signal from the transmitter 13, the transducer 2a generates an ultrasonic wave and converts a wave reflected from a subject into an electric signal (an echo signal). A matching layer is attached onto front sides of the plural transducers 2a in order to perform matching an acoustic impedance difference between the transducer 2a and the subject. A backing material is attached onto rear sides of plural transducers in order to prevent propagation of the ultrasonic wave. When the ultrasonic wave is transmitted from the transducer 2a to the subject, the ultrasonic wave is continuously reflected by discontinuous surfaces of acoustic impedances in a body tissue. The reflected ultrasonic wave is received as the echo signal by the transducer 2a. An amplitude of the echo signal depends on the acoustic impedance difference in the discontinuous surface that reflects the ultrasonic wave. When the ultrasonic wave is reflected by a surface of a blood stream, a heart wall, or the like, the echo signal is subjected to a frequency shift that depends on a velocity component in an ultrasonic transmission direction of a movable body by a Doppler effect.

The puncture needle 100 is a needle that is inserted in the subject. Typically, an adapter for the puncture needle 100 is attached to the ultrasonic probe 2. The adapter acts as a guide of the puncture needle 100. An operator inserts the puncture needle 100 in the subject through the adapter. All the needles, such as a needle used in biopsy and a needle used in RFA, which are inserted in the subject can be used as the puncture needle according to the embodiment.

The detector 4 detects a position at a leading end of the puncture needle 100, and generates data related to the detected position. The data related to the leading end of the puncture needle 100 is supplied to the scanning region determination unit 11.

The scanning region determination unit 11 determines a first scanning region for first ultrasonic scanning and a second scanning region for second ultrasonic scanning. Specifically, the scanning region determination unit 11 sets the first scanning region according to an instruction from the operator through the input unit 29. Typically, the first scanning region is set to a relatively wide three-dimensional region. The scanning region determination unit 11 determines the second scanning region based on the position at the leading end of the puncture needle 100, which is detected by the detector 4. A second scanning region determining method is roughly divided into two methods. In a first method, the second scanning region is set so as to include the position at the leading end of the puncture needle 100 in a substantial center of the second scanning region. In a second method, the second scanning region is set so as to include a predicted reachable position at the leading end of the puncture needle 100 in the substantial center of the second scanning region. The second scanning region is smaller than the first scanning region in a volume. Hereinafter, the first scanning region may be referred to as a wide scanning region and the second scanning region is referred to as a local scanning region.

The transmitter 13 includes a trigger generating circuit, a delay circuit, and a pulsar circuit, which are not illustrated in the drawings. The pulsar circuit repeatedly generates a rate pulse at a predetermined rate frequency fr Hz (period; 1/fr second) in order to form a transmitted ultrasonic wave. The delay circuit applies a delay time to each rate pulse in each channel according to a transmission direction and a transmission focal position. The trigger generating circuit applies the driving signal to the ultrasonic probe 2 at timing based on the rate pulse. An ultrasonic transmission beam, which is related to the transmission direction and the transmission focal position according to the delay time, is transmitted from the ultrasonic probe 2 by the application of the driving signal.

The transmitter 13 has a function of being able to instantaneously change a transmission frequency, a transmission driving voltage, and the like according to an instruction of the transmission/reception controller 17. Particularly, the change of the transmission driving voltage is implemented by a linear amplifier type of oscillation circuit that can instantaneously switch the transmission driving voltage or a mechanism that electrically switches plural power supply units.

The receiver 15 includes an amplifier circuit, an A/D converter, and a beam former, which are not illustrated in the drawings. The amplifier circuit amplifies the echo signal from the ultrasonic probe 2 in each channel. The A/D converter performs A/D conversion to the amplified echo signal. The beam former applies the delay time, which is necessary to determine a beam direction of an ultrasonic reception beam, to the digital echo signal in each reception focal position, and the beam former adds the echo signal to which the delay time is provided. A received signal corresponding to the ultrasonic reception beam is generated by the delay and addition.

The transmission/reception controller 17 controls the transmitter 13 and the receiver 15 in order to perform ultrasonic scanning according to the instruction from the operator through the input unit 29. Specifically, the transmission/reception controller 17 performs the first ultrasonic scanning for the wide scanning region and the second ultrasonic scanning for the local scanning region. The operator can arbitrarily set a imaging mode of the first ultrasonic scanning and a imaging mode of the second ultrasonic scanning through the input unit 29. All the existing imaging modes, such as a B mode, a Doppler mode, an elastography mode, a Wall Motion Tracking (WMT) mode, a contrast-enhanced mode, a spatial compound mode, a Shear Wave Elastography (SWE) mode, and a synthetic aperture mode, can be used to the imaging mode according to the embodiment. The transmission/reception controller 17 controls the transmitter 13 and the receiver 15 to switch between the first ultrasonic scanning for the wide scanning region and the second ultrasonic scanning for the local scanning region according to the instruction from the operator through the input unit 29.

The B-mode processor 19 performs logarithmic amplification and envelope detection processing to the received signal from the receiver 15, and generates B mode data in which a signal intensity is expressed by brightness. The B-mode processor 19 is actuated when the imaging mode of the ultrasonic scanning is the B mode. The B-mode data is supplied to the image generator 23.

The Doppler-mode processor 21 performs a frequency analysis to the received signal from the receiver 15, extracts the blood stream, the tissue, and a contrast medium echo component by the Doppler effect, and generates Doppler data in which pieces of blood stream information such as an average velocity, a dispersion, and a power, are expressed in color. The Doppler-mode processor 21 is actuated when the imaging mode of the ultrasonic scanning is the Doppler mode. The Doppler data is supplied to the image generator 23.

The image generator 23 generates ultrasonic image data corresponding to the imaging mode of the first ultrasonic scanning with respect to the wide scanning region, when the first ultrasonic scanning is performed based on the B-mode data from the B-mode processor 19 or the Doppler data from the Doppler processor 24. The image generator 23 generates ultrasonic image data corresponding to the imaging mode of the second ultrasonic scanning with respect to the local scanning region, when the second ultrasonic scanning is performed based on the B-mode data or the Doppler data. Specifically, the image generator 23 generates two-dimensional image data constructed by pixels or a volume data constructed by voxels based on the B-mode data or the Doppler data. The image generator 23 performs three-dimensional image processing based on the volume data, and generates the two-dimensional image data. For example, volume rendering, Multi Planar Reconstruction (MPR), and Maximum Intensity Projection (MIP) can be used as the three-dimensional image processing according to the embodiment. For example, the image generator 23 generates a B-mode image based on the B-mode data when the imaging mode is the B mode. The image generator 23 generates a Doppler image based on the Doppler data when the imaging mode is the Doppler mode. The image generator 23 generates an elasto image, in which a spatial distribution of hardness information on the subject is expressed, based on the Doppler data when the imaging mode is the elastography mode or the SWE mode. The image generator 23 generates a WMT image, in which a spatial distribution of motor function information on an organ is expressed, based on the Doppler data when the imaging mode is the WMT mode. The image generator 23 generates a contrast-enhanced image in which signal component from the ultrasonic contrast medium is specifically drawn when the imaging mode is the contrast-enhanced mode. The data before the data is supplied to the image generator 23 may be referred to as “raw data”.

The ultrasonic image data generated by the image generator 23 is stored in the storage unit 25. The control program for the ultrasonography according to the embodiment is stored in the storage unit 25.

The display unit 27 displays the ultrasonic image generated by the image generator 23 on a display device. For example, a CRT display, a liquid crystal display, an organic EL display, and a plasma display can be used as the display device.

An input device is mounted on the input unit 29 in order to take various instructions from the operator in the apparatus body 6. For example, the input unit 29 inputs an instruction to switch between the first ultrasonic scanning and the second ultrasonic scanning according to the instruction from the operator. For example, a trackball, various switches, a button, a mouse, and a keyboard can be used as the input device.

The system controller 31 has a function as an information processing device (a computer) to control the operation of the ultrasonic diagnostic apparatus 1. The system controller 31 reads the control program for the ultrasonography according to the embodiment from the storage unit 25, and controls each unit according to the control program.

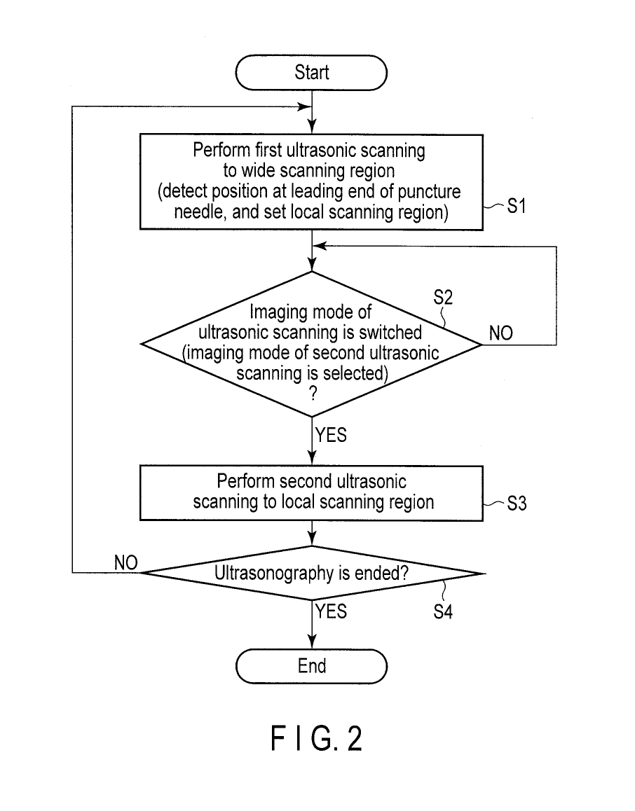

The ultrasonography according to the embodiment performed under the control of the system controller 31 will be described by taking needle biopsy performed under an ultrasonic guide as an example. FIG. 2 is a view illustrating a typical flow of the ultrasonography performed under the control of the system controller 31.

As illustrated in FIG. 2, the system controller 31 starts the ultrasonic scanning according to the first embodiment once the operator inputs a scanning start instruction through the input unit 29. During the ultrasonic scanning, the operator starts to insert the puncture needle for the needle biopsy into a target region in the subject.

The system controller 31 causes the transmission/reception controller 17 to perform the first ultrasonic scanning (Step S1). In Step S1, the transmission/reception controller 17 controls the transmitter 13 and the receiver 15 in order to perform the first ultrasonic scanning for the wide scanning region. The first ultrasonic scanning is used as a guide for the puncture needle to reach the target region. Accordingly, typically the imaging mode of the first ultrasonic scanning is set to the B mode in which morphological information on the subject can be drawn. The wide scanning region is set to the three-dimensional region. Before the first ultrasonic scanning is started, the scanning region determination unit 11 sets the wide scanning region according to the instruction from the operator through the input unit 29. In the B-mode scanning, under the control of the transmission/reception controller 17, the transmitter 13 transmits the ultrasonic wave from the ultrasonic probe 2 such that the wide scanning region is repeatedly scanned with the ultrasonic wave. The receiver 15 generates the received signal in each ultrasonic beam under the control of the transmission/reception controller 17. The B-mode processor 19 performs B-mode processing to the generated received signal to generate the B-mode data. The image generator 23 generates the volume data related to the wide scanning region based on the generated B-mode data, and generates a predetermined B-mode image based on the generated volume data. Typically, a sectional image related to an A section and a sectional image related to a B section can be cited as the B-mode image generated by the B-mode scanning in Step S1. The A section is a normal section, namely, an ultrasonic scanning surface, and the B section is a surface in which the A section is rotated by 90 degrees about a center axis (corresponding to a scanning line of a steering angle of 0 degree of the scanning surface). The operator inserts the puncture needle 100 toward the target region while observing the sectional image related to the A section and the sectional image related to the B section, which are displayed by the display unit 27.

The B-mode image generated in Step S1 is not limited to the sectional image related to the A section and the sectional image related to the B section. For example, the B-mode image may be a sectional image related to a section except the A section and the B section. The B-mode image is not limited to the sectional image, but the B-mode image may be a volume rendering image generated by the volume rendering or a projection image generated by pixel value projection processing.

During the ultrasonography, the detector 4 repeatedly performs the detection processing. The detector 4 detects the position at the leading end of the puncture needle in a real space. For example, the detector 4 is constructed by a position sensor attached to the leading end of the puncture needle 100. The position sensor is a sensor that can detect the position in the real space using magnetism or light. In this case, the detector 4 detects the position at the leading end of the puncture needle 100 at constant time intervals, and transmits the data of the detected position to the apparatus body 6. The system controller 31 causes the scanning region determination unit 11 to perform determination processing during the ultrasonography.

The method for detecting the position at the leading end of the puncture needle with the detector 4 is not limited to the method in which the position sensor is used. For example, the detector 4 may detect the position at the leading end of the puncture needle by performing image processing to the ultrasonic image drawn by the puncture needle. Specifically, the detector 4 can detect a leading end region of the puncture needle from the ultrasonic image using a brightness value the leading end of the puncture needle or a shape of the puncture needle. The detector 4 may be provided in the apparatus body 6 in the case that the position at the leading end of the puncture needle is detected through image processing.

In Step 1, the scanning region determination unit 11 determines the local scanning region based on the position at the leading end of the puncture needle 100, which is detected by the detector 4. The local scanning region may be either the two-dimensional shape or the three-dimensional shape as long as the volume of the local scanning region is smaller than that of the wide scanning region. The center of the local scanning region is aligned with the current position at the leading end of the puncture needle 100.

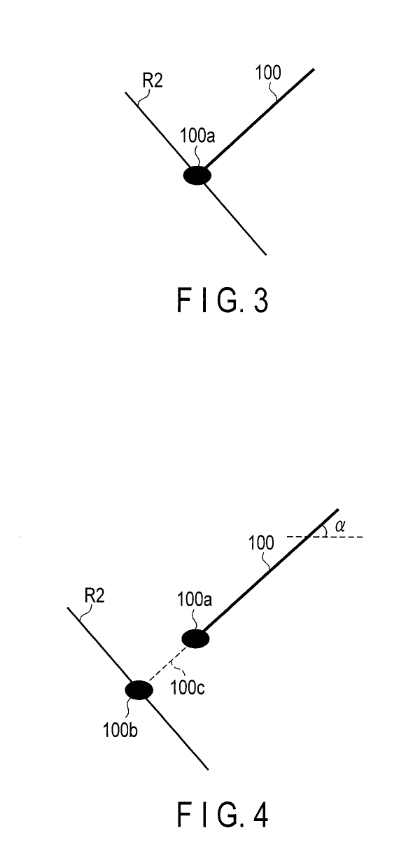

FIG. 3 is a view illustrating an example of a local scanning region R2 including the center of the current position at a leading end 100a of the puncture needle 100. As illustrated in FIG. 3, in the case that the local scanning region R2 is the section (scanning surface), the scanning surface is set so as to include the current position at the leading end 100a of the puncture needle 100 and so as to be orthogonal to the puncture needle 100.

As described above, the center of the local scanning region may be aligned with not the current position at the leading end of the puncture needle 100, but the predicted reachable position at the leading end of the puncture needle 100. FIG. 4 is a view illustrating an example of the local scanning region R2 including a center of a predicted reachable position 100b at the leading end 100a of the puncture needle 100. As illustrated in FIG. 4, in the case that the local scanning region R2 is the section (scanning surface), the scanning surface is set so as to include a predicted reachable position 100b at the leading end 100a of the puncture needle 100 and so as to be orthogonal to a predicted course 100c of the leading end 100a. The scanning region determination unit 11 determines the predicted reachable position 100b based on the current position at the leading end 100a, which is detected by the detector 4, and an inserting angle α of the puncture needle 100. The inserting angle α may be calculated by ant existing method. For example, the scanning region determination unit 11 calculates the inserting angle α based on positional data from the position sensor provided at the leading end 100a of the puncture needle 100 and positional data from a position sensor (not illustrated) provided in a base of the puncture needle 100. The scanning region determination unit 11 may calculate the inserting angle using the puncture needle region drawn on the ultrasonic image. The scanning region determination unit 11 calculates the predicted course 100c based on the current position of the leading end 100a and the inserting angle. The scanning region determination unit 11 determines a position, which is located on the predicted course 100c and is away from the current position of the leading end 100a by a predetermined distance, as the predicted reachable position 100c. The operator can arbitrarily set the predetermined distance through the input unit 29. The predicted course 100c may be calculated based on a locus of the position of the leading end 100a, which is detected by the detector 4.

The operator can arbitrarily adjust the size and the shape of the local scanning region through the input unit 29. The scanning region determination unit 11 updates the local scanning region in real time. That is, during the ultrasonography, the scanning region determination unit 11 can cause the local scanning region to follow the position at the leading end of the puncture needle 100. In other words, the scanning region determination unit 11 changes the position of the local scanning region in conjunction with the movement of the leading end of the puncture needle 100.

In Step S1, the display unit 27 may display the position at the leading end of the puncture needle 100, which is detected by the detector 4, while overlapping the position at the leading end of the puncture needle 100 with the ultrasonic image related to the first ultrasonic scanning. For example, a mark indicating the position at the leading end of the puncture needle 100 or an arrow indicating the position at the leading end of the puncture needle 100 may be overlapped.

When Step S1 is performed, the system controller 31 waits for the input unit 29 to switch the imaging mode (Step S2). When the puncture needle 100 reaches the target region, the operator checks a sample tissue for the purpose of the biopsy. The sample tissue is checked using the detailed morphological information, functional information, and the like. Accordingly, in the B-mode scanning of the wide scanning region, because the sample tissue near the leading end of the puncture needle cannot accurately be checked, the switching between the scanning regions and the switching between the imaging modes are performed in Step S2.

The operator presses a switching button, which is provided in the apparatus body or the like, to perform the switching between the scanning regions and the switching between the imaging modes. By pressing the switching button, the first ultrasonic scanning is switched to the second ultrasonic scanning, and the wide scanning region is switched to the local scanning region. The imaging mode of the second ultrasonic scanning may be different from or identical to the imaging mode of the first ultrasonic scanning. The imaging mode of the second ultrasonic scanning may previously be registered through the input unit 29, or the imaging mode of the second ultrasonic scanning maybe selected through the input unit 29 during the switching.

When the imaging mode is not switched in Step S2 (NO in Step S2), the system controller 31 repeats the first ultrasonic scanning.

When the imaging mode is not switched in Step S2 (YES in Step S2), the system controller 31 causes the transmission/reception controller 17 to perform the second ultrasonic scanning (Step S3). In Step S3, the transmission/reception controller 17 causes the transmitter 13 and the receiver 15 to repeatedly perform the second ultrasonic scanning for the local scanning region set by the scanning region determination unit 11. In performing the second ultrasonic scanning, the image generator 23 generates the ultrasonic image according to the second ultrasonic scanning, and the display unit 27 displays the generated ultrasonic image. During the second ultrasonic scanning, the detector 4 and the scanning region determination unit 11 are repeatedly actuated. That is, during the second ultrasonic scanning, the scanning region determination unit 11 causes the local scanning region to follow the position at the leading end of the puncture needle 100. The image generator 23 repeatedly generates the ultrasonic image including the position at the leading end of the puncture needle 100, which changes with time, or the predicted reachable position. The display unit 27 instantaneously displays the repeatedly-generated ultrasonic image. It is said that the ultrasonic image is an image in which a puncture line is a visual line while the position at the leading end of the puncture needle 100 or the predicted reachable position is a point of view. That is, the display unit 7 can provides a realistic sense as if the operator is located at the leading end of the puncture needle 100.

In Step S3, the display unit 27 may display the position at the leading end of the puncture needle 100, which is detected by the detector 4, or the predicted reachable position while overlapping the position at the leading end of the puncture needle 100 or the predicted reachable position with an ultrasonic image 12 related to the second ultrasonic scanning. For example, the mark indicating the position at the leading end of the puncture needle 100 or the arrow indicating the position at the leading end of the puncture needle 100 may be overlapped.

The second ultrasonic scanning will be described below. In the following description, the local scanning region can be applied to both the case that the current position at the leading end of the puncture needle 100 is included in the center of the local scanning region and the case that the predicted reachable position at the leading end of the puncture needle 100 is included in the center of the local scanning region. However, for the sake of convenience, it is assumed that the current position at the leading end of the puncture needle 100 is included in the center of the local scanning region.

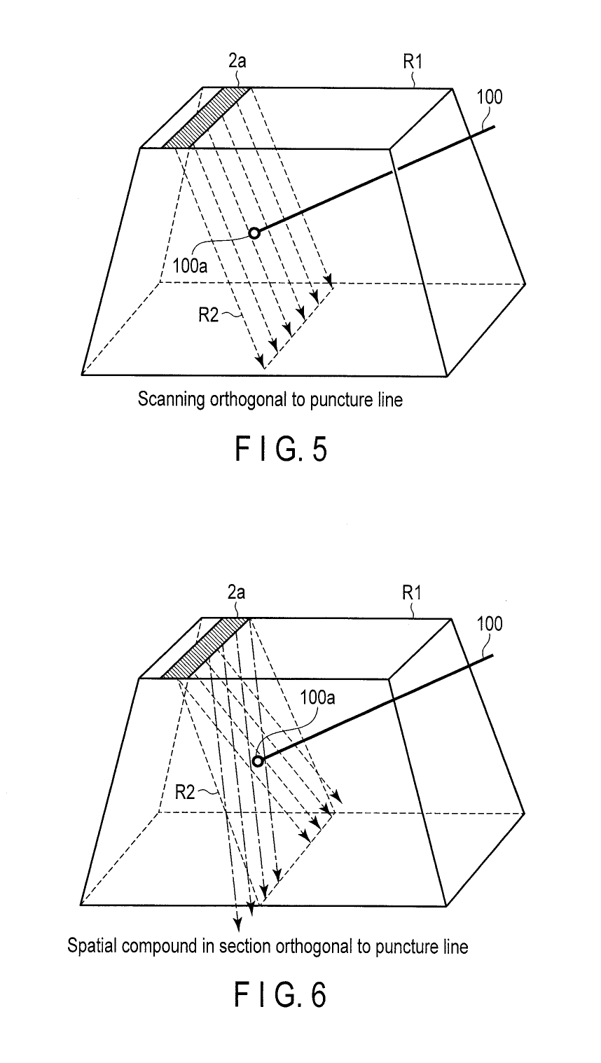

FIG. 5 is a view illustrating an example of the second ultrasonic scanning. As illustrated in FIG. 5, a wide scanning region R1 of the first ultrasonic scanning is set to a relatively wide three-dimensional region because the first ultrasonic scanning is used as the guide of the puncture needle 100. Therefore, a frame rate (time resolution) of the first ultrasonic scanning (the three-dimensional scanning) may be less real time. Spatial resolution of the sectional image based on the volume data is inferior to that of the B-mode image generated by the two-dimensional scanning.

On the other hand, as illustrated in FIG. 5, the local scanning region R2 of the second ultrasonic scanning is set to a relatively narrow range because the second ultrasonic scanning is used to observe the tissue near the leading end 100a of the puncture needle 100. For example, as illustrated in FIG. 5, the local scanning region R2 is set to the scanning surface (section) in which the center includes the position at the leading end 100a of the puncture needle 100. Thus, the time resolution and the spatial resolution of the second ultrasonic scanning can be improved better than those of the first ultrasonic scanning by restricting the scanning region to the two-dimensional region near the leading end of the puncture needle. For example, in order to improve visibility of the tissue near the leading end 100a of the puncture needle 100, the local scanning region R2 may be set so as to be orthogonal to the puncture line. At this point, an orientation of the scanning surface (local scanning region R2) is set based on the inserting angle of the puncture needle 100. The scanning surface (local scanning region R2) may be set to any angle with respect to the puncture line. The transmission/reception controller 17 selects the transducer 2a, which is used to transmit the ultrasonic wave, from the transducers mounted on the ultrasonic probe 2 according to the position and the orientation of the set local scanning region R2. The transmitter 13 transmits the ultrasonic wave to the local scanning region using the selected transducer 2a.

The local scanning region R2 is set to the section that is orthogonal to the puncture line near the leading end 100a of the puncture needle 100. However, in the embodiment, the local scanning region R2 is not limited to the section that is orthogonal to the puncture line near the leading end 100a of the puncture needle 100. For example, the local scanning region R2 may be a three-dimensional region near the leading end 100a or any plural sections including the puncture needle 100. The local scanning region R2 and the transducer 2a used to transmit the ultrasonic wave can follow each other in conjunction with the movement of the puncture needle 100.

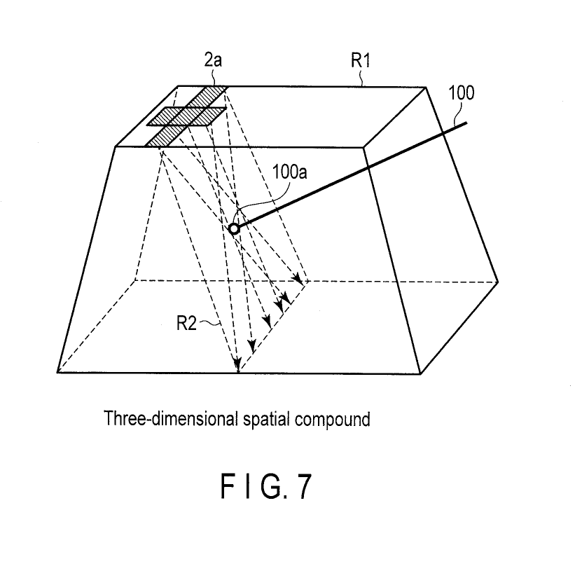

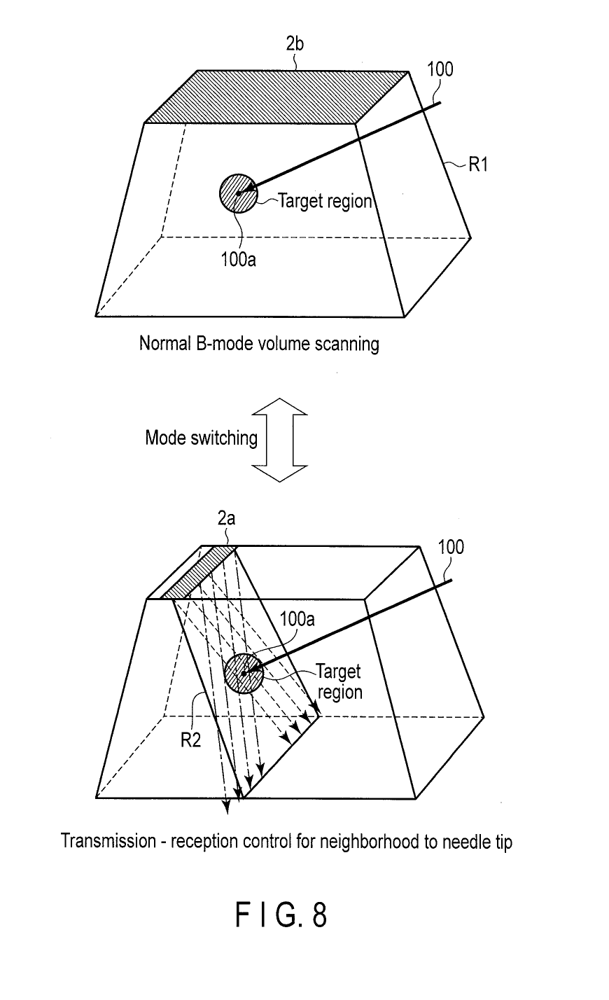

FIG. 6 is a view illustrating another example of the second ultrasonic scanning, and is a view illustrating the ultrasonic scanning performed to the two-dimensional local scanning region R2 using the two-dimensional spatial compound. In the spatial compound, the transmitter 13 performs deflection-scanning for the two-dimensional local scanning region (the scanning surface) R2 under the control of the transmission/reception controller 17. The transducers 2a used to transmit the ultrasonic wave are plural transducers, which intersect the local scanning region (scanning surface) R2 and are arrayed in line. FIG. 7 is a view illustrating another example of the second ultrasonic scanning, and is a view illustrating the ultrasonic scanning performed to the three-dimensional local scanning region R2 using the three-dimensional spatial compound. The transducers 2a used to transmit the ultrasonic wave are plural two-dimensionally-arrayed transducers including one-line transducers that intersect the local scanning region (scanning surface) R2 and transducers that are adjacent to the one line transducers.

As illustrated in FIGS. 6 and 7, the second ultrasonic scanning may be the ultrasonic scanning in which the spatial compound is used. The plural ultrasonic waves corresponding to plural transmission directions are transmitted to the two-dimensional local scanning region by the deflection-scanning. The image generator 23 generates the B-mode image related to the local scanning region R2 with respect to each of the plural ultrasonic waves. The image generator 23 synthesizes the plural B-mode images corresponding to the transmissions of the plural ultrasonic waves, and generates a single B-mode image (a synthetic image). The display unit 27 displays the generated synthetic image. In the synthetic image, image quality is improved by the effect of the spatial compound compared with the B-mode image of the ultrasonic scanning related to the single transmission direction. Accordingly, the ultrasonic scanning in which the spatial compound is used is performed as the second ultrasonic scanning, which allows the operator to observe the tissue near the leading end of the puncture needle as the high-quality ultrasonic image.

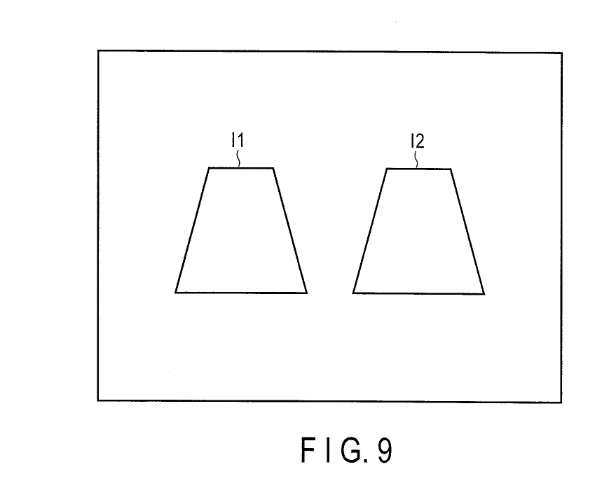

As described above, the transmission/reception controller 17 switches between the first ultrasonic scanning and the second ultrasonic scanning according to the instruction from the operator through the input unit 29. FIG. 8 is a view schematically illustrating the switching between the first ultrasonic scanning and the second ultrasonic scanning. As illustrated in FIG. 8, for example, the normal B-mode volume scanning is applied as the first ultrasonic scanning, and the localized B-mode scanning is applied to the neighborhood of the leading end 100a of the puncture needle 100 as the second ultrasonic scanning. In the first ultrasonic scanning, all the transducers 2b included in the ultrasonic probe 2 are used to transmit the ultrasonic waves. In the second ultrasonic scanning, as described above, some transducers 2a in the ultrasonic probe 2 are used to transmit the ultrasonic waves. The input unit 29 includes a user interface (U/I) that instantaneously switches between the first ultrasonic scanning and the second ultrasonic scanning. The operator operates the user interface, whereby the transmission/reception controller 17 instantaneously switches between the normal B-mode volume scanning and the localized scanning for the neighborhood of the leading end 100a.

FIG. 9 is a view illustrating a display example of an image in the ultrasonography. As illustrated in FIG. 9, in performing the second ultrasonic scanning, the display unit 27 displays an ultrasonic image I1 related to the first ultrasonic scanning and ultrasonic image I2 related to the second ultrasonic scanning while the ultrasonic image I1 and the ultrasonic image 12 are located side by side. The ultrasonic image I1 is an image generated during the first ultrasonic scanning, and is a still image. For example, the ultrasonic image I1 is the image based on the volume data, which is generated by the image generator 23 through the B-mode volume scanning. The sectional image related to the A section and the like, the volume rendering image by the volume rendering, the projection image by the Maximum Intensity Projection (MIP) are suitable to the method for displaying the ultrasonic image I1. One sectional image may be displayed, or plural sectional images, such as three sections orthogonal to one another, may be displayed. The ultrasonic image I2 is an image generated in real time, and is a moving image. For example, the ultrasonic image I2 is the B-mode image related to the two-dimensional local scanning region, which is generated by the image generator 23 through the B-mode scanning. Accordingly, the ultrasonic image I2 is superior to the ultrasonic image I1 in the time resolution and the spatial resolution. In the second ultrasonic scanning, the method for displaying the ultrasonic image I2 is not limited to the above method. For example, the ultrasonic image I2 may be displayed while overlapped with the ultrasonic image I1. Alternatively, only the ultrasonic image I2 may be displayed.

The imaging mode of the second ultrasonic scanning is not limited to one kind. For example, plural kinds of imaging modes may be set as the imaging mode of the second ultrasonic scanning. That is, the imaging mode of the second ultrasonic scanning may be at least two kinds in the B mode, the Doppler mode, the elastography mode, the Wall Motion Tracking (WMT) mode, the contrast-enhanced mode, the SWE mode, the spatial compound mode, and the synthetic aperture mode. In the second ultrasonic scanning, the plural kinds of ultrasonic scanning corresponding to the plural kinds of imaging modes are alternately repeated every predetermined number of times of the ultrasonic wave transmission and reception under the control of the transmission/reception controller 17. In this case, the ultrasonic image I2 in FIG. 9 is an image in which the ultrasonic image related to the first imaging mode and the ultrasonic image related to the second imaging mode are overlapped with each other. For example, in the second ultrasonic scanning, the B-mode scanning and the Doppler-mode scanning may alternately be repeated. In this case, the ultrasonic image I2 is an image in which the B-mode image and the Doppler image are overlapped with each other. More particularly, in the ultrasonic image I2, the B-mode image is updated during the B-mode scanning, and the Doppler image is updated during the Doppler-mode scanning. This display enables the operator to simultaneously observe the morphological information and the blood stream information in real time.

For the purpose of the improvement of the image quality, various pieces of image processing may be performed to the ultrasonic image I2 related to the second ultrasonic scanning. For example, the micro structure extracting processing described in Patent Literature 2 can be applied to the ultrasonic image I2 based on the spatial compound. In the case of a mammary clinical application, a tissue may be sampled in a fine calcified region to diagnose benignancy/malignancy in the pathological examination. Whether the puncture needle is inserted in the desired calcified region is hardly checked only by the B-mode volume scanning. However, the checking is easily performed by applying the above processing to the neighborhood of the needle tip. It is well known that an ability of extracting the micro structure included in the ultrasonic image I2 is improved by applying the image processing described in Patent Literature 2 to the ultrasonic image I2 based on the spatial compound.

The imaging mode of the second ultrasonic scanning is not limited to the B mode. As described above, the elastography mode, the Doppler mode, the contrast-enhanced mode, and the like can also be applied to the imaging mode of the second ultrasonic scanning. For example, in the case of the elastography mode, the operator presses the subject using the ultrasonic probe 2, and releases the subject. The Doppler-mode processor 19 calculates a spatial distribution of tissue velocity information caused by the press and the release. The image generator 23 calculates a spatial distribution of hardness information based on the spatial distribution of the calculated velocity information, and generates an elastography image in which the spatial distribution of the calculated hardness information is expressed in color. The display unit 27 displays the generated elastography image.

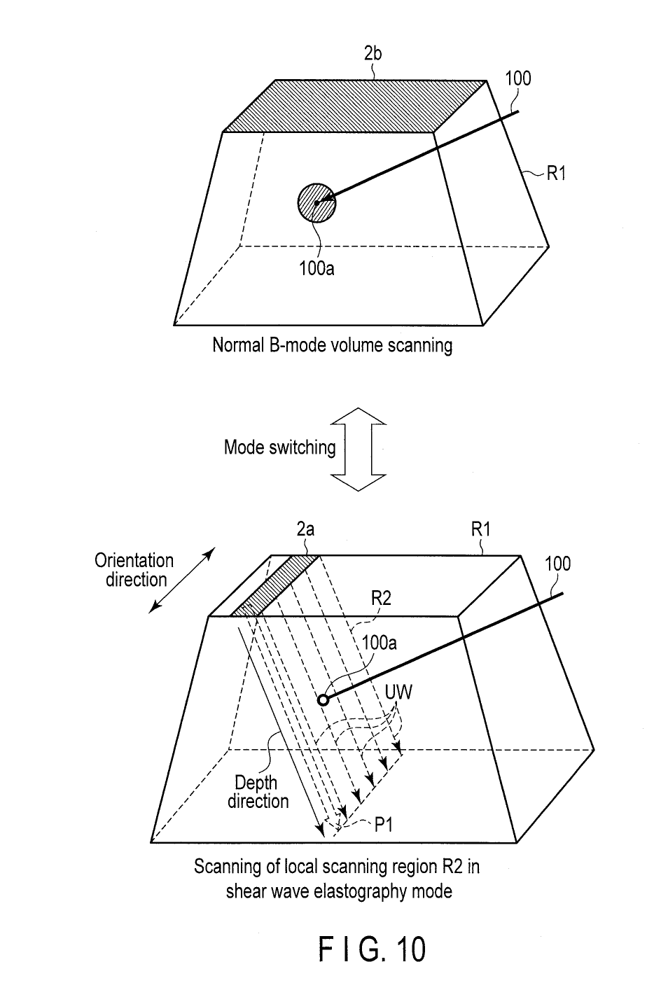

The imaging mode of the second ultrasonic scanning may be the SWE mode. The SWE mode is an imaging method in which a phenomenon, in which a propagation velocity of a shear wave in the scanning region depends on the hardness of the tissue, is used. The case that the second ultrasonic scanning is the SWE mode will be described in detail with reference to FIG. 10. In performing the first ultrasonic scanning, the operator inputs an instruction to switch the first ultrasonic scanning for the second ultrasonic scanning through the input unit 29. In response to the input of the switching instruction, the system controller 31 switches the first ultrasonic scanning for the second ultrasonic scanning. In the second ultrasonic scanning, the transmission/reception controller 17 performs SWE scanning for the local scanning region.

The local scanning region may be either the section or the three-dimensional region. However, in the following description, it is assumed that the local scanning region is the section.

The transmission/reception controller 17 causes the transmitter 13 to transmit a high-pressure ultrasonic pulse P1 called a push pulse. Specifically, the transmitter 13 transmits the push pulse P1, which is focused on a predetermined transmission focal position, to an end portion with respect to the orientation direction of the local scanning region R2. The transmitter 13 repeatedly transmits the push pulse P1 while switching the transmission focal position along a depth direction. The shear wave is generated in the local scanning region R2 when the push pulse P1 is transmitted. The shear wave is a transverse wave. The shear wave propagates in the local scanning region R2 to deform the tissue in a local scanning region R1. A deformation degree of the tissue by the shear wave depends on the tissue hardness.

When the push pulse P1 is transmitted, the transmission/reception controller 17 performs a shear wave propagation measuring mode. Specifically, the transmission/reception controller 17 controls the transmitter 13 to repeatedly transmit a tracking pulse UW over the whole local scanning region R2 in order to measure the shear wave, and the transmission/reception controller 17 controls the receiver 15 to repeatedly receive the ultrasonic wave from the local scanning region R2. More particularly, the transmission/reception controller 17 repeatedly transmits and receives the tracking pulse UW plural times to and from an observation region T1 that is away from the transmission position of the push pulse P1 by a predetermined distance L1. The observation region T1 is a partial region of the local scanning region R2. The tracking pulse UW is an ultrasonic pulse that is used to observe a displacement amount of the tissue in the observation region T1 and a time the displacement is generated. The Doppler-mode processor 21 repeatedly calculates a spatial distribution of the tissue displacement with respect to the observation region T1 by performing an autocorrelation calculation to the received signal from the receiver 15. The image generator 23 calculates a spatial distribution of a shear wave reaching time with respect to the observation region T1 based on the spatial distributions of the tissue displacements with respect to the different times. The shear wave reaching time corresponds to a time the displacement amount of the tissue from a reference time becomes the maximum. For example, the reference time is defined by a time the push pulse is transmitted.

When transmitting the tracking pulse UW to the position away from the push pulse transmission position by the predetermined distance L1, the transmission/reception controller 17 transmits the push pulse P1 again, and repeatedly transmits and receives the tracking pulse UW to and from an observation region T2 that is away from the push pulse transmission position by a predetermined distance L2. The observation region T2 is a partial region of the local scanning region R2. Therefore, the image generator 23 calculates the spatial distribution of a shear wave reaching time with respect to the observation region T2 like the scanning performed to the observation region T1.

When the tracking pulse UW is transmitted to the whole local scanning region R2, the image generator 23 generates the SWE image in which the tissue hardness is expressed in color. At this point, it is well known that there is a constant proportional relationship between the shear wave propagation velocity and the tissue hardness. That is, the region where the shear wave propagation velocity is high is a high-elasticity, hard region. The region where the shear wave propagation velocity is low is low-elasticity, soft region. According to the proportional relationship, the image generator 23 calculates a spatial distribution of the tissue hardness based on the spatial distribution of the shear wave reaching time with respect to the local scanning region R2. The image generator 23 generates the SWE image in which the tissue hardness is expressed in color. The display unit 27 displays the SWE image.

In the above operation, the transmission of the push pulse and the transmission and reception of the tracking pulse are sequentially performed to the observation regions while the observation region is deviated. However, there may be various variations of the operation. For example, what is called a simultaneous reception technique in which plural receiving beams are formed with respect to the one transmitting beam such that the observation region is spread may be used for the tracking pulse.

In order to improve the accuracy of the SWE image, the synthetic image may be displayed based on the two SWE images related to the different push pulse transmission positions. In this case, the transmission/reception controller 17 alternately repeats the first SWE scanning and the second SWE scanning by the similar method. That is, in the first SWE scanning, the transmission/reception controller 17 controls the transmitter 13 to transmit the push pulse to one end portion of the local scanning region R2, and controls the transmitter 13 and the receiver 15 to perform the scanning in a shear wave propagation measuring mode to the whole local scanning region R2. The Doppler-mode processor 21 performs the autocorrelation calculation to the received signal from the receiver 15 to calculate the spatial distribution of the tissue displacement, and the image generator 23 generates the first SWE image based on the spatial distribution of the tissue displacement. In the second SWE scanning, the transmission/reception controller 17 controls the transmitter 13 to transmit the push pulse to the other end portion of the local scanning region R2, and controls the transmitter 13 and the receiver 15 to perform the scanning in the shear wave propagation measuring mode to the whole local scanning region R2. The Doppler-mode processor 21 performs the autocorrelation calculation to the received signal from the receiver 15 to calculate the spatial distribution of the tissue displacement, and the image generator 23 generates the second SWE image based on the spatial distribution of the tissue displacement.

The first SWE scanning and the second SWE scanning are alternately repeated. When the first SWE image and the second SWE image are generated, the image generator 23 generates the synthetic image of the first SWE image and the second SWE image. The display unit 27 displays the synthetic image. The operator can more correctly evaluate the tissue hardness by observing the synthetic image.

In the above SWE scanning, it is assumed that the local scanning region is the section. Alternatively, in the SWE scanning of the embodiment, the local scanning region may be the three-dimensional region.

When Step S3 is performed, the system controller 31 waits for an instruction to end the ultrasonic scanning from the operator through the input unit 29 (Step S4). When Step S3 is performed, the operator observes the ultrasonic image by the second ultrasonic scanning, and determines whether the leading end of the puncture needle reaches the target region. When determining that the leading end of the puncture needle reaches the target region, the operator samples the tissue and the like. When the biopsy is ended, the operator inputs an instruction to end the ultrasonography through the input unit 29.

When the instruction to end the ultrasonography is input in Step S4 (YES in Step S4), the system controller 31 ends the ultrasonography.

The clinical application example of the embodiment is not limited only to the needle biopsy. The embodiment can be applied to the radio frequency ablation of the localized tumors, such as the liver cancer, namely, all the ultrasonic diagnoses, such as the RFA treatment, in which the puncture needle is used. The puncture needle (electrode needle), to which an electrode is attached in order to generate a high temperature in a surface of the puncture needle, is used in the RFA treatment. More particularly, the RFA treatment is a treatment method, in which the electrode needle is inserted from the body surface into the tumor portion and a pathological change portion is coagulated by the high temperature generated by the radio wave. Nowadays, the contrast-enhanced ultrasonic wave is frequently used in an RFA treatment effect determination. This is because the blood stream is rich by a blood vessel before the treatment while cancer cells are destroyed to decrease the blood stream after the treatment. Although real-time three-dimensional ultrasonic scanning is useful to determine the treatment effect of the three-dimensionally-distributed tumors, the real-time three-dimensional ultrasonic scanning is inferior to two-dimensional ultrasonic scanning in the time resolution and the spatial resolution.

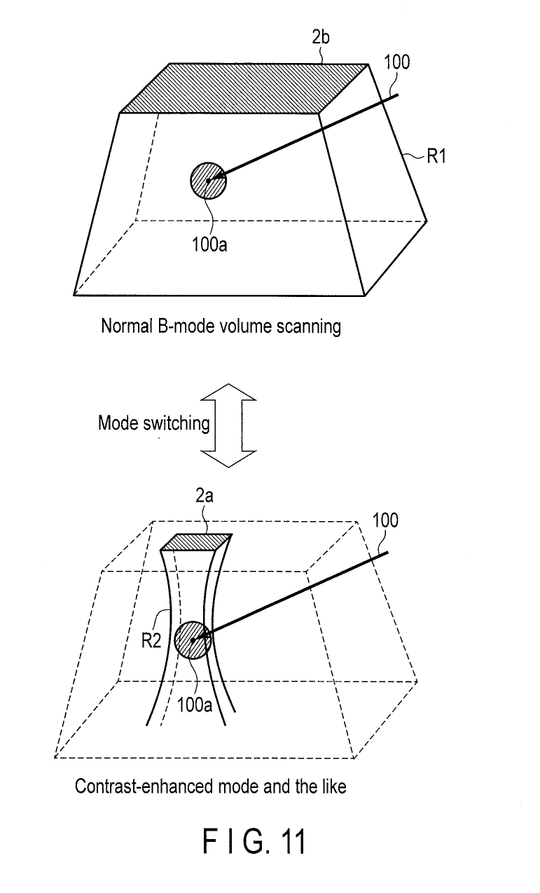

In the case of the RFA treatment, the electrode needle is inserted toward the treatment region in Step S1 in FIG. 2. When the electrode needle reaches the target region, the treatment region is ablated by the electrode needle. For example, the electrode needle is connected to a treatment apparatus. An output intensity and an output time of the treatment apparatus are set according to a tumor size and a kind of the RFA needle. The imaging mode of the second ultrasonic scanning is selected in Step S2. For example, the contrast-enhanced mode, the Doppler mode, and the elastography mode are selected as the imaging mode. The imaging mode suitable to observe the decrease of the tumor blood stream by the treatment and the change in hardness associated with tissue degeneration is selected. Particularly, in the contrast-enhanced mode, it is necessary to observe a temporal change of inflow of the contrast-enhanced medium. However, because of the contrast-enhanced mode localized only to an interest region, the high time resolution can be maintained, but the real-time characteristic is not sacrificed. The multi-section display and the volume display can properly be selected as the display method. As illustrated in FIG. 11, the B-mode three-dimensional scanning and the contrast-enhanced-mode scanning for the local scanning region can instantaneously be switched. In Step S3, the treatment is performed in the imaging mode selected in Step S2. Whether the blood stream is left in the tumor portion is observed. As a result of the observation, the treatment is ended when the treatment is performed to the enough range. When the tumor portion is left, the flow returns to Step S1, the electrode needle is inserted or the additional treatment is performed. Accordingly, the embodiment implements the improvement of the accuracy of the RFA treatment effect determination.

As described above, the ultrasonic diagnostic apparatus 1 of the embodiment provides a technology useful for the ultrasonography in which the puncture needle is used.

Modifications according to the embodiment will be described below.

(First Modification)

It is assumed that the local scanning region according to the embodiment is determined based on the current position at the leading end of the puncture needle 100 or the predicted reachable position corresponding to the current position. In this case, the local scanning region moves in conjunction with the movement of the current position at the leading end of the puncture needle 100. The puncture needle 100 may move carelessly even if the target region should be observed during the needle biopsy or the RFA treatment. In the case that the local scanning region is determined in conjunction with the current position at the leading end of the puncture needle 100, the local scanning region deviates from the target region when the puncture needle 100 is pulled out from the observation object regions, such as the target region. Accordingly, it is necessary for the operator to adjust the position of the puncture needle 100 every time the puncture needle 100 deviates from the observation object region. The local scanning region according to a first modification is determined based on the previously-stored past position at the leading end of the puncture needle 100. An ultrasonic diagnostic apparatus and an ultrasonic imaging method according to the first modification will be described below. In the following description, a component having the substantially same function as the embodiment is designated by the same numeral, and the overlapping description is made as needed basis.

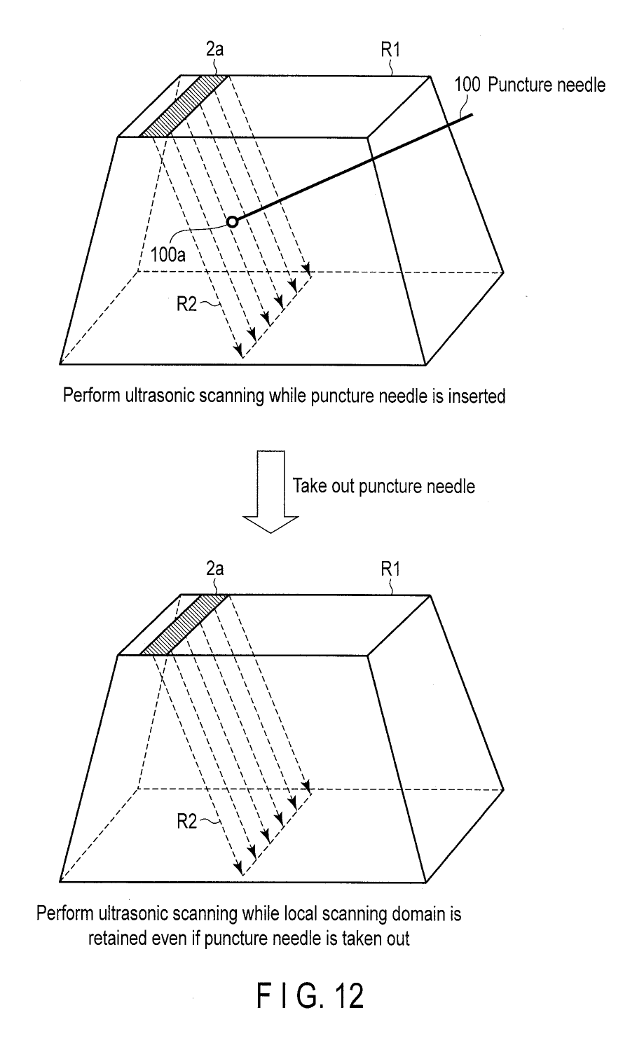

FIG. 12 is a view illustrating local scanning region determining processing performed according to the first modification. As illustrated in FIG. 12, in inserting the puncture needle 100, the local scanning region is set in conjunction with the position at the leading end of the puncture needle 100. The operator inputs a storage instruction through the input unit 29 when determining that the leading end 100a of the puncture needle 100 reaches the position where the local scanning region R2 should be fixed. For example, the operator inputs the storage instruction when the leading end of the puncture needle 100 reaches the observation object region. In response to the input of the storage instruction, the positional data of the leading end 100a at a time point the storage instruction is input is stored in the storage unit 25. The scanning region determination unit 11 reads the positional data stored in the storage unit 25, and determines the local scanning region based on the read position. Therefore, even if the puncture needle 100 moves, the local scanning region does not move in conjunction with the position of the leading end 100a, but the local scanning region is fixed. In the case that the operator inputs an interlocking instruction through the input unit 29, the scanning region determination unit 11 causes the local scanning region R2 to interlock with the current position at the leading end of the puncture needle 100.

In the above description, it is assumed that, in response to the input of the storage instruction, the local scanning region R2 is instantaneously set to the positional data stored in the storage unit 25 to perform the second ultrasonic scanning. However, the embodiment is not limited to the above description. That is, the storage of the local scanning region R2 and the ultrasonic scanning may separately be performed. For example, when the operator inputs the fixing instruction through the input unit 29, the scanning region determination unit 11 reads the positional data of the leading end 100a, which is stored in the storage unit 25, and determines the local scanning region R2 based on the read position. In this case, the scanning region determination unit 11 can set the local scanning region R2 to the observation object region even after the puncture needle 100 is pulled out from the observation object region.

Accordingly, even if the position at the leading end of the puncture needle 100 and the observation object region are deviated from each other, a trouble of inserting the puncture needle 100 again to observe the observation object region can be eliminated.

In the case that the second ultrasonic scanning is the SWE mode, the ultrasonic diagnostic apparatus of the embodiment can perform the SWE scanning while the puncture needle 100 is pulled out from the local scanning region. Accordingly, the improvement of the accuracy of the SWE mode is implemented in the first modification.

(Second Modification)

It is assumed that the local scanning region of the embodiment includes the position at the leading end of the puncture needle 100 or the predicted reachable position at the leading end. However, the local scanning region of the embodiment is not limited to this assumption. The local scanning region according to a second modification is determined based on the predicted course based on the current position at the leading end of the puncture needle 100. An ultrasonic diagnostic apparatus and an ultrasonic imaging method according to the second modification will be described below. In the following description, a component having the substantially same function as the embodiment is designated by the same numeral, and the overlapping description is made as needed basis.

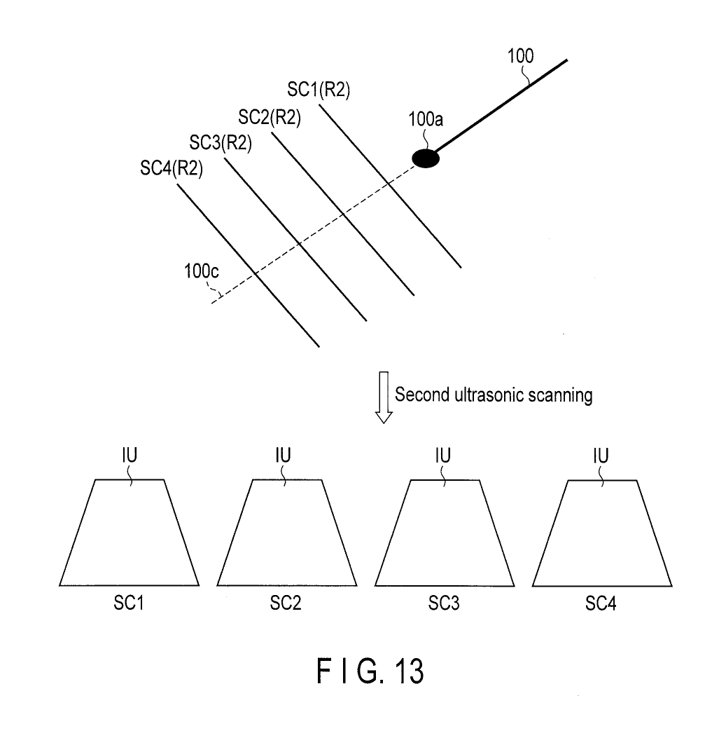

FIG. 13 is a view illustrating a method for determining the local scanning region R2 according to the second modification. It is assumed that the local scanning region R2 according to the second modification is a section SC. As illustrated in FIG. 13, during or before the needle biopsy, the scanning region determination unit 11 calculates the predicted course 100c of the puncture needle 100. Because the predicted course calculating method is described above, the description is omitted. When the predicted course 100c is calculated, the scanning region determination unit 11 sets plural sections SC orthogonal to the predicted course 100c to plural local scanning regions R2, respectively. For example, four sections SC1, SC2, SC3, and SC4 orthogonal to the predicted course 100c are set as illustrated in FIG. 13.

In response to the instruction to switch the first ultrasonic scanning for the second ultrasonic scanning, the transmission/reception controller 17 sequentially perform the second ultrasonic scanning for the plural local scanning regions SC (R2). Therefore, the image generator 23 generates the plural ultrasonic images IU related to the plural local scanning regions SC (R2), and the display unit 27 displays the plural ultrasonic images IU. The plural ultrasonic images IU may be displayed side by side, or the plural ultrasonic images IU may be displayed in the order from the side close to the leading end of the puncture needle 100.

In the second modification, before the insertion of the puncture needle 100, the inserting course of the puncture needle 100 can previously be observed by displaying the plural ultrasonic images orthogonal to the predicted course of the puncture needle 100.

Accordingly, the inserting course of the puncture needle 100 can be reviewed before the insertion of the puncture needle 100, and the re-insertion of the puncture needle 100 can be prevented.

[Effect]

As described above, the ultrasonic diagnostic apparatus 1 according to the embodiment includes at least the ultrasonic probe 2, the transmitter 13, the receiver 15, the detector 4, the scanning region determination unit 11, and the transmission/reception controller 17. The detector 4 detects the position at the leading end of the puncture needle in the real space. The scanning region determination unit 11 sets the wide scanning region in the subject, and sets the local scanning region narrower than the first scanning region based on the position at the leading end. The transmission/reception controller 17 controls the transmitter 13 and the receiver 15 to switch between the first scanning region corresponding to the wide scanning region and the second ultrasonic scanning corresponding to the local scanning region according to the instruction from the operator.

According to the above configuration, the ultrasonic diagnostic apparatus 1 can arbitrarily switch between the first ultrasonic scanning in which the puncture needle is guided to the target region and the second ultrasonic scanning in which the tissue information on the target region near the leading end of the puncture needle is checked in detail. Additionally, because the second ultrasonic scanning has the relatively narrow scanning region, the target region can be observed at the relatively high spatial resolution and the relatively high time resolution.

According to the embodiment, the ultrasonic diagnostic apparatus and the ultrasonic scanning method, for being able to improve accuracy of the ultrasonography in which the needle inserted in the subject is used, can be provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a transducer configured to generate an ultrasonic wave and converts the ultrasonic wave from a subject into an echo signal;
a transmitter configured to supply a driving signal to the transducer;
a receiver configured to perform signal processing to the echo signal from the transducer;
a detector configured to detect a position at a leading end of a puncture needle;
a determination unit configured to determine a first scanning region in the subject and a second scanning region based on the position at the detected leading end, the second scanning region being narrower than the first scanning region; and
a transmission/reception controller configured to control the transmitter and the receiver to switches between first ultrasonic scanning for the first scanning region and second ultrasonic scanning for the second scanning region according to an instruction from an operator.

2. The ultrasonic diagnostic apparatus according to claim 1, further comprising:
a generator configured to generate a first ultrasonic image related to the first scanning region based on an output signal from the receiver when the first ultrasonic scanning is performed, and the generator generating a second ultrasonic image related to the second scanning region based on the output signal from the receiver when the second ultrasonic scanning is performed; and
a display unit configured to display the first ultrasonic image and the second ultrasonic image.

3. The ultrasonic diagnostic apparatus according to claim 2, wherein the display unit displays the position at the detected leading end on the second ultrasonic image.

4. The ultrasonic diagnostic apparatus according to claim 2, wherein the display unit displays the first ultrasonic image and the second ultrasonic image while overlapping the first ultrasonic image and the second ultrasonic image with each other, or the display unit displays the first ultrasonic image and the second ultrasonic image while arraying the first ultrasonic image and the second ultrasonic image.

5. The ultrasonic diagnostic apparatus according to claim 1, wherein the second scanning region is a three-dimensional space or a two-dimensional section.

6. The ultrasonic diagnostic apparatus according to claim 1, wherein the first ultrasonic scanning is a B mode.

7. The ultrasonic diagnostic apparatus according to claim 1, wherein the second ultrasonic scanning is a spatial compound.

8. The ultrasonic diagnostic apparatus according to claim 1, wherein the second ultrasonic scanning is an elastography mode, a Doppler mode, a contrast-enhanced mode, or a shear-wave elastography mode.

9. The ultrasonic diagnostic apparatus according to claim 1, wherein the determination unit causes the second scanning region to track the leading end of the puncture needle.

10. The ultrasonic diagnostic apparatus according to claim 1, wherein the determination unit adjusts a range of the second scanning region according to the instruction from the operator.

11. The ultrasonic diagnostic apparatus according to claim 1, wherein the second ultrasonic scanning is at least two imaging modes in a B mode, a Doppler mode, an elastography mode, a contrast-enhanced mode, a spatial compound, and a shear-wave elastography mode, and
the transmission/reception controller alternately repeats the at least two imaging modes every predetermined number of times of ultrasonic wave transmission and reception.

12. The ultrasonic diagnostic apparatus according to claim 1, wherein the determination unit sets the second scanning region such that the position at the detected leading end is included in a substantial center of the second scanning region.

13. The ultrasonic diagnostic apparatus according to claim 1, wherein the determination unit sets the second scanning region such that a predicted reachable position of the detected leading end is included in a substantial center of the second scanning region.

14. The ultrasonic diagnostic apparatus according to claim 1, further comprising a storage unit configured to store the position of the detected leading end,
wherein the determination unit reads the position at the stored leading end from the storage unit according to the instruction from the operator, and the determination unit sets the second scanning region such that the position at the read leading end is included in a substantial center of the second scanning region.

15. The ultrasonic diagnostic apparatus according to claim 1, wherein the determination unit determines a plurality of sections orthogonal to a predicted course of the detected leading end as a plurality of second scanning regions, respectively.

16. The ultrasonic diagnostic apparatus according to claim 15, further comprising: a generator; and a display unit,
wherein the transmission/reception controller controls the transmitter and the receiver to scan each of the plurality of second scanning regions with the ultrasonic wave,
the generator generates a plurality of ultrasonic images related to the plurality of second scanning regions based on an output signal from the receiver, and
the display unit displays the plurality of ultrasonic images.

17. An ultrasonic scanning method comprising:
supplying a driving signal to a transducer incorporated in an ultrasonic probe;
performing signal processing to an echo signal from the transducer;
detecting a position at a leading end of a puncture needle attached to the ultrasonic probe;
determining a first scanning region in a subject and a second scanning region based on the position at the detected leading end, the second scanning region being narrower than the first scanning region; and
switching between first ultrasonic scanning for the first scanning region and second ultrasonic scanning for the second scanning region according to an instruction from an operator.
